(19) United States
(12) Patent Application Publication
(10) Pub. No.: US 2022/0211904 A1
(43) Pub. Date: Jul. 7, 2022

(54) POLY(GLYCEROL SEBACATE) URETHANE ELASTOMERIC LEAFLET FOR HEART VALVE REPLACEMENT

(71) Applicant: THE SECANT GROUP, LLC, Telford, PA (US)

(72) Inventors: Stephanie REED, Conshohocken, PA (US); Peter D. GABRIELE, Frisco, TX (US); Andrew METZGER, Lafayette Hill, PA (US); Amanda K. WEBER, Macungie, PA (US); Alexander M. STAHL, Haverford, PA (US)

(21) Appl. No.: 17/646,960

(22) Filed: Jan. 4, 2022

Related U.S. Application Data
(60) Provisional application No. 63/134,396, filed on Jan. 6, 2021.

(51) Int. Cl. A61L 27/18; A61L 27/34; A61L 27/50; C08G 18/42; C08J 5/18

(52) U.S. Cl. CPC A61L 27/18; A61L 27/34; A61L 27/505; C08G 18/4236; B29C 67/246; A61L 2430/20; A61L 2420/02; C08J 2375/06; C08J 5/18

(57) ABSTRACT

A leaflet for a heart valve replacement includes poly(glycerol sebacate) urethane. A heart valve replacement includes a leaflet including poly(glycerol sebacate) urethane. A process forms a leaflet for a heart valve replacement. The process includes casting a first solution including poly(glycerol sebacate) and isocyanate to form a leaflet composition including poly(glycerol sebacate) urethane. The process also includes shaping the leaflet composition to form the leaflet.

### RELATED APPLICATIONS

This application claims the benefit of and priority to U.S. Provisional Patent Application No. 63/134,396 filed Jan. 6, 2021, which is hereby incorporated by reference in its entirety.

### FIELD

The present disclosure is generally directed to heart valve replacements. More specifically, the present disclosure is directed to an elastomeric leaflet including poly(glycerol sebacate) urethane (PGSU) for a human heart valve replacement.

### BACKGROUND

Properly working human heart valves permit one-way flow of blood through the heart valve. Artificial heart valves may be used to replace damaged or defective human heart valves.

Leaflets in replacement human heart valves are conventionally mostly composed of xenogenic tissue, such as bovine or porcine pericardial tissue. These animal tissues require the heart valve product be stored in preservatives, such as glutaraldehyde or formaldehyde solutions, prior to implantation. These animal tissues also require the heart valve product to be stored under cold conditions, such as 4° C.

Attempts have been made to create synthetic leaflets using non-biodegradable polymers. One such example is the urethane-based leaflets of Foldax Inc. (Salt Lake City, Utah) (see, for example, U.S. Pat. No. 9,301,837, issued Apr. 5, 2016).

Attempts have also been made to create synthetic leaflets using biodegradable polymers, with the goal of regenerating the leaflet concurrently during polymer degradation. One such example is the lumen heart valve technologies of CorMatrix Cardiovascular, Inc. (Roswell, Ga.) that incorporate biodegradable polymers including poly(glycerol sebacate) (PGS) (see, for example, U.S. Patent App. Pub. No. 2019/0380831, published Dec. 19, 2019). PGS, poly(glycolic acid) (PGA), polylactic acid (PLA), poly(lactic-co-glycolic acid) (PLGA), polycaprolactone (PCL), and other biodegradable polyesters require refrigeration or frozen storage, and so the heart valve products containing such polymers also require such cold storage.

Endocarditis, an infection of the inner lining of the heart, is a significant issue after heart valve replacement.

The aforementioned synthetic, biodegradable polymer options cannot be stored at room temperature prior to implantation and lack the appropriate flexibility and compatibility to serve as a leaflet in an artificial heart valve.

### SUMMARY

In exemplary embodiments, a leaflet for a heart valve replacement includes poly(glycerol sebacate) urethane.

In exemplary embodiments, a heart valve replacement includes a leaflet including poly(glycerol sebacate) urethane.

In exemplary embodiments, a process forms a leaflet for a heart valve replacement. The process includes casting a first solution including poly(glycerol sebacate) (PGS) and isocyanate to form a leaflet composition including poly(glycerol sebacate) urethane (PGSU). The process also includes shaping the leaflet composition to form the leaflet.

In exemplary embodiments, a process forms a heart valve replacement. The process includes providing a heart valve replacement substrate. The process also includes applying a solution including poly(glycerol sebacate) and isocyanate to form a coating overlying at least a portion of at least one component of the heart valve replacement substrate, by a process selected from the group consisting of casting, spray coating, dip coating, electrospinning, and electrospraying. The coating includes poly(glycerol sebacate) urethane.

### BRIEF DESCRIPTION OF THE DRAWINGS

FIG. 1 schematically shows an artificial heart valve in an embodiment of the present disclosure.

FIG. 2 schematically shows a top view of a tricuspid valve.

FIG. 3 schematically shows a top view of a bicuspid valve.

Where possible, the same reference numbers are attempted to be used throughout the drawings to represent the same parts.

### DETAILED DESCRIPTION OF EXEMPLARY EMBODIMENTS

Provided are synthetic heart valve leaflets including poly(glycerol sebacate) urethane (PGSU).

Embodiments of the present disclosure, for example, in comparison to concepts failing to include one or more of the features disclosed herein, provide synthetic heart valve leaflets that are shelf stable, that do not require storage in a fixative, that do not require cold storage, that can be stored at room temperature and room humidity, that offer mechanical resilience to cyclic fatigue, that demonstrate bendability, foldability, and abrasion resistance during transcatheter loading and deployment followed by elastic springback and recovery once deployed to the intended anatomical site, that achieve predetermined mechanical properties and degradation profiles by tuning PGSU chemistry, that are fluid impermeable, that are biocompatible, that are hemocompatible, that are non-fibrotic, that have anti-adherent and/or non-fouling zones, that have pro-cellular migration zones, that have regenerative potential, and/or combinations thereof.

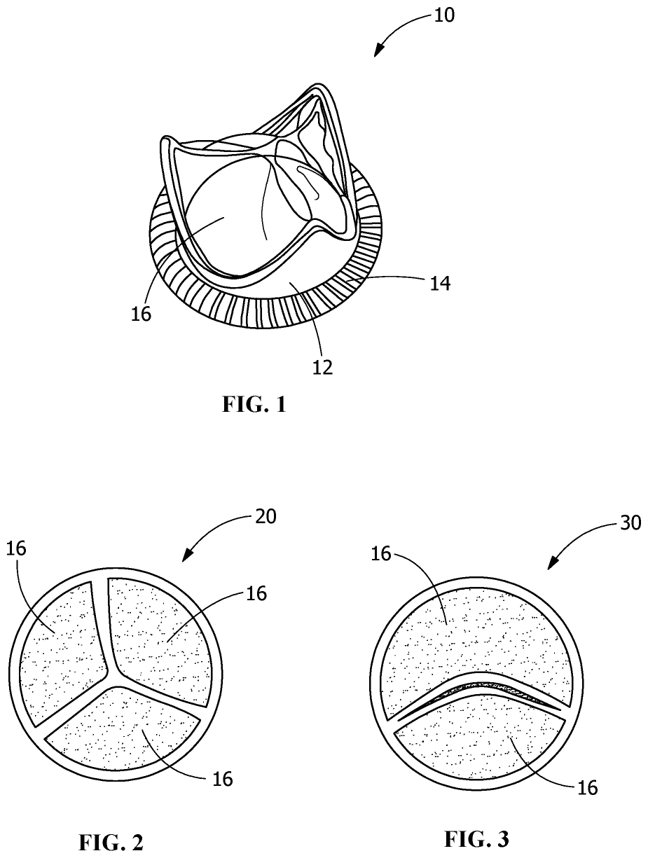

FIG. 1 schematically shows a heart valve replacement 10. The heart valve replacement 10 includes a heart valve frame 12 including an annulus ring 14. The heart valve replacement 10 also includes leaflets 16 extending from the heart valve frame 12 that covers the valve opening in the valve frame 12. The leaflets 16 open in a first direction to permit blood flow through the valve opening of the heart valve replacement 10. The leaflets 16 remain closed to flow, however, in a second direction opposite the first direction to prevent backflow of blood through the heart valve replacement 10.

The valve of the heart valve replacement 10 may include any suitable number of leaflets 16. In some embodiments, the heart valve replacement 10 includes a tricuspid valve or a bicuspid valve. FIG. 2 schematically show a top view of a tricuspid valve 20 having three leaflets 16. FIG. 3 schematically show a top view of a bicuspid valve 30 having two leaflets 16.

In exemplary embodiments, the leaflet 16 is a synthetic leaflet. The synthetic leaflet may form part of any heart valve replacement 10. The synthetic leaflet provides the heart valve function of opening to let blood move forward through the heart during one part of the heartbeat and closing to prevent backflow during another part of the heartbeat. The synthetic leaflet design may be for a bicuspid heart valve or a tricuspid heart valve. In some embodiments, the bicuspid heart valve is a mitral valve. In some embodiments, the tricuspid heart valve is an aortic valve, a pulmonary valve, or a tricuspid valve. In some embodiments, the synthetic leaflet is part of a mitral valve. In some embodiments, the synthetic leaflet is part of a tricuspid valve. In some embodiments, the synthetic leaflet is part of a pulmonary valve. In some embodiments, the synthetic leaflet is part of an aortic valve.

In exemplary embodiments, the leaflet 16 is shelf stable. As used herein, shelf stable refers to being storable without significant degradation detrimentally impacting product performance for at least 6 months at room temperature and room humidity without cold storage at 4° C. or below and without storage in a fixative.

The desirable properties of PGS resin, such as its hyperbranched architecture and high polydispersity, e.g., having a polydispersity index of at least 6, lead to highly bendable mechanical properties when crosslinked into PGSU elastomer. PGS resin synthesized through a water-mediated process may also result in compositions including a particular distribution of low and medium molecular weight oligomers, e.g., having a molecular weight below 3000 Da, that act as a plasticizer and aid flexure and elasticity.

PGSU formulated in the particular crosslinking range described here does not demonstrate substantial elongation under tension, for example less than two-fold elongation prior to break, with a typical strain at break of about 65%, meaning the strength under tension is fairly weak. However, the bending properties are excellent, for example bending 180 degrees and still exhibiting 100% elastic springback, meaning the elastic resilience to flexure is highly recoverable. This flexural resilience is good for heart valve leaflets, which undergo millions of cycles of flexing throughout a patient's lifetime when implanted within the aortic, mitral, or bicuspid valves. The elastomeric nature of PGSU is expected to also offer improved loading and deployment during transcatheter delivery and placement at the valve site, in particular when compared to common biodegradable polymer choices, such as other biodegradable polyesters like lactides, glycolides, and caprolactones.

PGSU formulated at 2.5:1 to 3.5:1 PGS:hexamethylene diisocyanate (HDI) w:w ratio crosslinking, solvent-free and solvated at 15-80% concentrations using acetone and propyl acetate as solvents, at 150-350 μm thicknesses, yielded mechanical properties of 7-26 MPa elastic modulus, 3-15 MPa ultimate tensile strength, resilience to creep upon tensile cycling, and 180-degree flexure upon 3-point bending.

PGSU formulated in the particular crosslinking range described here is efficiently crosslinked, with very little sol content, for example around 1% w/w sol. This leads to a thermoset PGSU with very few extractables and leachables, for example less than 1.5% w/w under exhaustive extraction conditions, and very little risk of unreacted polymer fractions blooming to the surface. Preventing blooming from occurring at room temperature and room humidity extends the shelf life. If blooming does occur in PGSU, such as PGSU formulations with lower crosslinking, unreacted sol content slowly travels to the surface of the thermoset part, often within 1 to 6 months shelf storage. This event causes opacity and rough surface texture on the part. It also causes stiffening of the elastomeric part, since the bloomed polymer fractions were previously acting as a plasticizer within the part, providing elasticity. PGSU formulated in the particular crosslinking range described here and using the particular PGS resin described here achieves high flexibility and elasticity without needing a large sol content to act as a plasticizer.

Reducing the extractable and leachable content also improves the biocompatibility of PGSU. The highly crosslinked nature of the PGSU formulations described herein also means fewer free functional groups are present within the polymer network and on the surface of the polymer part. Free functional groups such as carboxylates and hydroxyls are known to incite various inflammatory responses. PGSU with 3.5:1 PGS:HDI w:w ratio passes testing for cytotoxicity, acute systemic toxicity, irritation, sensitization, material mediated pyrogen, chromosomal aberration, Ames mutagenic activity, short-term 4-week implantation, and long-term 6-month implantation in both in vitro and in vivo evaluations, with scores of 0 and performance statistically comparable to negative controls, indicating no biocompatibility concerns. Importantly for cardiovascular devices, PGSU with 3.5:1 PGS:HDI w:w ratio passes testing for hemolysis, complement activation, partial thromboplastin time, and platelet and leukocyte adhesion, with scores of 0 and performance statistically comparable to negative controls, indicating no concerns about thrombus formation or blood interaction. PGSU with 3.5:1 and 2:1 PGS:HDI w:w ratios have also been implanted long-term for 3, 6, and 8 months in rat, dog, and cow animal models, without any fibrous encapsulation, foreign body giant cells, or residual lymphocytes or macrophages that would indicate a chronic inflammatory response at the implant site. Taken together, PGSU formulations described herein are well suited for the hemodynamic environment heart valve leaflets experience.

PGSU is a polymer with high enough crosslinking that it does not have shelf life stability or biocompatibility concerns, yet it still demonstrates full elastic recovery upon flexure.

In exemplary embodiments, PGSU leaflets are expected to outperform conventional biodegradable polymer materials, such as lactides glycolides, and caprolactones, due, in part, to the anti-microbial nature of PGS chemistry. PGSU leaflets may also outperform conventional biodegradable polymer materials due to the regenerative and pro-healing nature of PGS chemistry, for example through activation of M2 type macrophages or upregulation of pro-healing genetic markers.

In some embodiments, the PGS resin, from which the PGSU is formed, is prepared via a water-mediated polycondensation reaction, such as described in U.S. Pat. No. 9,359,472, which is hereby incorporated by reference herein, which copolymerizes glycerol and sebacic acid. In some embodiments, the PGS resin for crosslinking to PGSU is a chemically-characterized PGS resin, as described in U.S. Patent Application Publication No. 2020/0061240, which is hereby incorporated by reference herein.

In exemplary embodiments, the chemical characterization of the PGS resin may include, but is not limited to, one or more of the following parameters: its molecular weight, its polydispersity index, its acid number, its hydroxyl number, and/or its stoichiometric ratio of glycerol-to-sebacic acid. One or more of these PGS resin parameters may be controlled to tailor the PGSU mechanical properties and to tailor the PGSU degradation rate to achieve desired tissue ingrowth and regeneration. Additionally, the isocyanate-to-hydroxyl stoichiometric ratio to form the PGSU from the PGS resin may be controlled to tailor the PGSU for use in a synthetic leaflet 16.

The chemically-characterized PGS resin may include a weight average molecular weight above 10,000 Da, alternatively above 15,000 Da, alternatively in the range of 14,000 Da to 20,000 Da, alternatively in the range of 15,000 Da to 18,000 Da, alternatively above 25,000 Da, or any value, range or sub-range therebetween.

The chemically-characterized PGS resin may include a polydispersity index of at least 6, alternatively at least 8, alternatively in the range of 6 to 16, alternatively in the range of 8 to 12, alternatively in the range of 8 to 10, alternatively less than 16, alternatively less than 14, alternatively less than 12, alternatively less than 10, alternatively less than 8, or any value, range, or sub-range therebetween.

The chemically-characterized PGS resin may include an acid number between 20 and 80, alternatively between 30 and 70, alternatively between 40 and 60, alternatively between 35 and 55, alternatively between 35 and 45, alternatively between 40 and 50, or any value, range, or sub-range therebetween.

The chemically-characterized PGS resin may include a hydroxyl number between 160 and 240, alternatively between 180 and 220, alternatively between 190 and 210, or any value, range, or sub-range therebetween. As used herein, a “hydroxyl number” value is as determined by American Society for Testing and Materials (ASTM) E222.

The chemically-characterized PGS resin may include a stoichiometric ratio of glycerol-to-sebacic acid between 1:0.25 and 1:2, alternatively between 1:0.5 and 1:1.5, alternatively between 1:0.75 and 1:1.25, alternatively about 1:1, or any value, range, or sub-range therebetween.

The PGSU may be formulated with a stoichiometric ratio of isocyanate-to-hydroxyl between 1:0.25 and 1:2, alternatively between 1:0.25 and 1:1.5, alternatively between 1:0.25 and 1:1.25, or any value, range, or sub-range therebetween.

In some embodiments, a relative amount of isocyanate is selected with respect to PGS to provide an isocyanate-to-hydroxyl stoichiometric ratio in the range of 1:10 to 4:1, alternatively in the range of 1:10 to 1:4, alternatively in the range of 1:4 to 1:3, alternatively in the range of 4:3 to 5:2, alternatively in the range of 1:2 to 4:1, alternatively in the range of 1:1 to 4:1, alternatively in the range of 4:5 to 4:1, alternatively in the range of 2:3 to 4:1, or any value, range, or sub-range therebetween. Selection of an appropriate isocyanate-to-hydroxyl stoichiometric ratio may provide a stable implantable product with optical clarity that does not exhibit clouding, hazing, blooming, or stiffening over time upon storage at room temperature and room humidity ambient conditions. When the isocyanate-to-hydroxyl stoichiometric ratio is 1:2 or less (for example, 1:2, 1:3, or 1:4), the implantable product may suffer from clouding and stiffening when stored at ambient conditions, which may be detrimental to product shelf life and may reflect an unstable product. An isocyanate-to-hydroxyl stoichiometric ratio in the range of 4:5 to 4:1 has been observed to provide a long shelf life under ambient storage conditions.

In exemplary embodiments, a PGS resin with a weight average molecular weight in the range of 14,000 Da to 20,000 Da demonstrates a high degree of branching and high polydispersity at a 1:1 glycerol-to-sebacic acid stoichiometric ratio, and such PGS resin results in a PGSU with elasticity, flexure, long-term degradation, and shelf life especially suitable for good leaflet performance. Long-term degradation may mean a lifespan greater than 6 months or alternatively greater than 12 months following implantation prior to complete bioerosion.

In exemplary embodiments, the isocyanate is a diisocyanate crosslinker. Appropriate isocyanates may include, but are not limited to, HDI, methylene diphenyl diisocyanate (MDI), toluene diisocyanate (TDI), isophorone diisocyanate (IPDI), methylenebis(cyclohexyl isocyanate) (HMDI), tetramethylxylene diisocyanate (TMXDI), aliphatic isocyanates, aromatic isocyanates, aliphatic-aromatic combination isocyanates, and/or blocked isocyanates. Different isocyanates may impart different degrees of flexibility, such as, for example, linear isocyanates and isocyanates of increasing chain length may provide greater flexibility. In some embodiments, the isocyanate is HDI. In some embodiments, an amount of isocyanate is selected to provide a PGS:isocyanate w:w mass ratio in the range of 2:1 to 4:1, alternatively about 3.5:1, or any value, range, or sub-range therebetween.

In some embodiments, the core composition formed by combining the liquid PGS composition and the liquid isocyanate composition also includes a catalyst. The catalyst may be included in the liquid PGS composition or the liquid isocyanate composition. Appropriate catalysts may include, but are not limited to, catalysts containing metals and/or catalysts of metal salts, such as, for example, tin (II) 2-ethylhexanoate. Appropriate catalysts may contain or include, but are not limited to, tin, platinum, caffeine, potassium, sodium, calcium, magnesium, citric acid, citrate in salt form, such as, for example, potassium citrate, tartaric acid, and/or tartrate in salt form, such as, for example, potassium tartrate. In some embodiments, the catalyst is tin (II) 2-ethylhexanoate and an amount of catalyst is selected to provide a PGS:tin mass ratio in the range of 20:1 to 2000:1, alternatively 200:1 to 1600:1, alternatively about 800:1, or any value, range, or sub-range therebetween.

In exemplary embodiments, at least a portion of a synthetic leaflet of a heart valve replacement is made of PGSU. In some embodiments, the synthetic leaflet includes a coating of PGSU on a substrate. In other embodiments, the PGSU forms the synthetic leaflet, such as, for example, as a standalone film.

In some embodiments, a coating including PGSU is applied on a leaflet substrate material to form the synthetic leaflet. In some embodiments, the composition of the PGSU in the coating is selected to improve the seal of the synthetic leaflet when the synthetic leaflet is in a closed position. PGSU is a water-impermeable and blood-tight polymer that may be applied to or may form any or all of the leaflet, skirt, or other heart valve component. Perivalvular leakage can occur around the annulus of a heart valve, and PGSU sealant or coating may reduce leakage at this location. Leakage or backflow can also occur at the interface where leaflets meet, and PGSU leaflet designs and formulations may be tuned to exhibit greater flexibility and self-adhesive properties at the leaflet edges, compared to the leaflet base, which may be tuned to exhibit greater stiffness and anti-adhesive properties. In exemplary embodiments, the PGSU is elastomeric and unexpectedly exhibits high self-adhesion. The flexibility and self-adhesive properties of PGSU can be tuned by changing the PGSU crosslinking density, tackiness, coefficient of friction, surface finish, surface energy, polymer chain alignment, functional group arrangement, hydrogen bonding, van der Waals interactions, ionic interactions, non-specific binding, non-covalent interactions, and/or geometry, such as, for example, the leaflet thickness. Such a design may allow leaflets to stay sufficiently strong to open and close robustly while also demonstrating resilience to suction and back pressure.

In some embodiments, the PGSU coating is applied on a textile to form a leaflet. The textile may be biodegradable or non-biodegradable.

In some embodiments, the PGSU coating is applied to entirely cover the underlying textile topography, yielding a smooth or other patterned surface. In some embodiments, the PGSU-coated textile may demonstrate isotropy in architecture and mechanical properties. In some embodiments, the PGSU-coated textile may demonstrate anisotropy in architecture and mechanical properties.

In some embodiments, the PGSU coating is applied on a pyrolytic carbon leaflet in a mechanical heart valve.

In some embodiments, the PGSU coating is applied on a xenogenic leaflet. It will be appreciated however, that some presently preferred embodiments are free of any xenogenic materials.

In some embodiments, the PGSU coating is applied on a synthetic polymer leaflet substrate material. The synthetic polymer may be biodegradable or non-biodegradable.

In other embodiments, the leaflet includes a tie coat of PGS or PGSU to adhere PGSU onto an underlying leaflet substrate material.

In some embodiments, a PGSU coating is applied with a thickness in the range of about 1 μm to 500 μm. In some embodiments, the PGSU coating is applied by a spray coating technique. In other embodiments, the PGSU coating is applied by a dip coating technique. In some embodiments, the PGSU coating is applied by flowing through or around the substrate. In such embodiments, the substrate may have a two-dimensional form, such as, for example, a sheet or a three-dimensional form, such as, for example, a tube.

In other embodiments, a PGSU coating or standalone PGSU film is applied by a draw down bar process. In other embodiments, a PGSU coating or standalone PGSU film is applied by blade coating process or air knife coating process.

For PGSU coatings and/or standalone PGSU films, the PGS resin may be neat in a solvent-free formulation or solvated in an organic solvent. Appropriate organic solvents may include, but are not limited to, acetone, propyl acetate, ethyl acetate, ethanol, isopropyl alcohol, dichloromethane (DCM), tetrahydrofuran (THF), or combinations thereof.

The physiochemical properties of PGS and PGSU formulations in their liquid state prior to cure may aid in the successful manufacture of a leaflet product including PGSU. More specifically, the materials and compositions may be selected to provide a predetermined viscosity, surface tension, and/or surface energy for the wetting and subsequent delamination from release liners used in processing. PGS and PGSU formulations in their liquid state also exhibit non-Newtonian behavior such as shear thinning, which impacts process parameters like flow rate and draw down rate.

For spray coating, PGS solution and isocyanate solution may be pumped through separate lines that meet and co-mingle at an atomizing surface of an ultrasonic mixing nozzle, in which the intermixed PGS-isocyanate droplets are then sprayed out as a mist onto substrates. For spray coating, an appropriate liquid state may include a 1-25% w/w PGS solution. Appropriate spray coating parameters may include 0.5 mL/min flow rate, 600-1500 mm/min speed of nozzle translation, 1-60 passes by the nozzle over the substrate, 1-24 hours drying time in between depositing layers, and/or 24 hours curing and drying time to finish the final product. For spray coating, appropriate release liner substrate may include polyester films, for example, polyethylene terephthalate (PET).

For dip coating, drawing down, blade coating, air knife coating, flowing, casting, molding, and/or overmolding, PGS and isocyanate may be pre-mixed together into a single PGSU solution with sufficient working time for processing. For these processes, an appropriate liquid state may include 5-95% w/w PGSU solutions and solvent-free PGSU. For dip coating, appropriate withdrawal rates may be in the range of 10-5000 mm/min.

For drawing down, a 380-635 μm draw down bar height achieves PGSU films having a thickness in the range of 150-350 μm. Appropriate draw down rates may be in the range of 300-1500 mm/min. For drawing down, appropriate release liner substrates may include polyester films such as PET.

For cast or molded parts, such as standalone PGSU films, an appropriate liquid state may include 15-80% w/w PGS solutions. In exemplary embodiments, PGSU sheets or films are cast with a thickness ranging from 100 μm to 1 cm.

In some embodiments, a textured, roughened, marbled, patterned, or channeled release liner or substrate imparts surface textures and designs to PGSU after it is delaminated. In some embodiments, such surface features are anisotropic, to better mimic the native leaflet structure, to selectively impart certain functionality, and/or to selectively encourage specific cell and/or tissue type ingrowth in specific zones. Alternatively, in some embodiments, such surface features are isotropic.

In some embodiments, a textile is co-laminated onto one side of a PGSU cast film by incorporating the textile into the casting process while the PGSU is still liquid and uncured. In some embodiments, the textile is permitted to float to the top of the liquid. In other embodiments, the textile is secured to the bottom of the mold.

In other embodiments, the textile is embedded within the center of a PGSU cast film by incorporating the textile into the casting process while PGSU is still liquid and uncured. In some embodiments, the textile is submerged in the liquid and remains in the center naturally due to PGSU viscosity. In other embodiments, the textile is secured in the center of the mold.

In other exemplary embodiments, the synthetic leaflet of a heart valve replacement is constructed solely of PGSU. These embodiments may be coated or seeded with compositions containing or more synthetic or autogenic components and, in some embodiments, the entirety of the synthetic leaflet consists of PGSU.

In some embodiments, PGSU is cast or molded into sheets, which are then cut to dimension to form the synthetic leaflet. Appropriate processes for cutting the sheets may include, but are not limited to, laser cutting, electrical discharge machining (EDM), hot knife, die cutting, stamping, razor, scissors, or sharp blade.

For cast or molded parts, PGS resin may be used neat in a solvent-free formulation or solvated in an organic solvent. Appropriate solvents may include, but are not limited to, acetone, propyl acetate, ethyl acetate, ethanol, isopropyl alcohol, DCM, THF, or combinations thereof. For cast or molded parts, such as PGSU sheets, 20-80% w/w PGS solutions work well.

In exemplary embodiments, homogeneous mixing provides uniform PGSU crosslinking and correspondingly uniform biodegradation. Homogeneity can be achieved using static or dynamic mixing of the polyol and isocyanate components. Beads or pins may be included to achieve better incorporation of the two immiscible materials of disparate viscosity.

In some embodiments, the PGSU casting is formed by an open mold casting process. In some embodiments, however, PGSU open mold casting processes are sensitive to moisture in the air-facing interface, which can be mitigated by performing the casting in a nitrogen blanket or moisture-free or moisture-controlled environment.

PGSU open mold casting processes may be sensitive to polymer chain alignment and functional group orientation changes at the air-facing interface. In some embodiments, moisture exposure and polymer chain alignment are both mitigated by placing a thin polymer film to cover the air-facing interface and then peeling the thin film away after curing is complete. One appropriate thin polymer film is a PET release liner. Minimizing the air-facing interface by casting PGSU with release liners on both sides, in this sandwich approach, can also lead to smooth surfaces on both sides of the PGSU film. This approach is also amenable to imparting different patterns or degrees of texture to one surface of PGSU versus the other, for example by using an embossed release liner that has a predetermined texture replicating in the surface of the PGSU film.

PGSU open mold casting processes, when PGS resin is solvated within the formulation, may be sensitive to the emergence of an orange-peel surface texture, which results from a mismatch between solvent evaporation and PGSU curing phenomena and timing, and which, like mitigation for moisture exposure and chain alignment, can also be mitigated by placing a thin polymer film, such as a PET release liner, to cover the air-facing interface and then peeling the thin film away after curing is complete. This can also be mitigated with careful solvent selection for vapor pressure to aid in timing solvent evaporation relative to PGSU curing. This can also be mitigated by the selection of the percent solids content in the PGS solution. In exemplary embodiments, a combination of a ratio of a higher vapor pressure solvent and a lower vapor pressure solvent and a percent solids content is selected to coordinate the solvent evaporation rate to be similar to the rate of PGSU cure. For example, acetone has a vapor pressure of 185.5 mm Hg at 20° C., whereas propyl acetate has a vapor pressure of 25 mm Hg at 20° C., and various blends of the two solvents can carefully tune the formulation solvent evaporation rate behavior to better synchronize with PGSU curing times, to prevent emergence of orange-peel surface roughness, and/or to avoid any undesirable surface or coating effects. PGSU set-to-touch time may be about 1 minute to about 1 day, alternatively about 1 minute, alternatively about 5 minutes, alternatively about 1 hour, alternatively about 12 hours, alternatively about 24 hours, or any value, range, or sub-range therebetween. PGSU time to complete curing may be about 5 minutes to about 1 day, alternatively about 5 minutes, alternatively about 1 hour, alternatively about 12 hours, alternatively about 24 hours, or any value, range, or sub-range therebetween. Previously discussed 5-95% w/w PGSU solutions or 20-80% w/w PGS solutions work well for open mold casting with acetone and propyl acetate co-solvent blends.

PGSU thin films fabricated by draw down bar processes may be susceptible to rough surface textures developing in the top surface caused by uneven dragging of uncured prepolymer mixture, especially with the use of more viscous prepolymer mixtures. This effect can be mitigated by selecting an appropriate draw down speed that is slow enough to allow the PGS resin to relax and flow beneath the draw down bar smoothly, but fast enough that the resin is drawn evenly and does not build up behind the draw down bar. This effect may also be mitigated by careful solvent selection, with small amounts of solvent enabling the resin solution to flow more smoothly, while still holding their shape once drawn.

The surface energy of the substrate used for film fabrication by casting, molding, spray-coating, or draw down bar processes may impact film thickness and uniformity. Low surface energy substrates may cause films to bead up, disrupting the continuity or even thickness of the PGSU material. In exemplary embodiments, suitable substrates have sufficiently high surface energy to enable wetting of the prepolymer mixture. In exemplary embodiments, however, the substrate does not interact too tightly with the cured PGSU, as this can interfere with the successful release of intact films. Examples of substrates appropriate for PGSU film fabrication may include, but are not limited to, PET and aluminum sheeting.

Exposure to moisture, whether in the air-facing interface or in raw materials, such as PGS resin, isocyanate, catalyst, or solvents, may cause the formation of urea once the isocyanate is exposed to water. In exemplary embodiments, this is mitigated by drying PGS resin, such as to below 0.1% water content, such as by placing in an oven at 60° C. and 10 torr for 24 hours. PGS resin may alternatively be dried at the end of the synthesis process inside the reactor.

In some embodiments, the PGSU casting is formed by a closed mold casting process. Closed molding PGSU eliminates air-facing interface issues.

In some embodiments, the closed molds include runner designs that promote co-mingling of material and homogeneous mixing. Molds may include designs with few 90-degree bends, since the viscosity of PGSU is very high, to reduce the pressure inside the mold and the clamp force needed to keep the mold closed during filling. Molds may be filled using reaction injection molding equipment or meter-mix-dispense equipment, such as those commonly used for silicone and polyurethane dispensing. Molds may be made of aluminum or stainless steel to achieve good wetting of PGSU as it flows through the mold, and also good delamination, release, and demolding of PGSU once cured. In exemplary embodiments, the mold material is selected to have an appropriate surface energy to ensure good wetting and good demolding of the PGSU. Higher surface energy substrates have been demonstrated to provide good performance. Molds may also be made of certain polymers, such as polyethylene (PE), PET, or silicone.

In some embodiments, the PGSU is provided in a fiber form that is then processed as a yarn, where the yarn is woven, knitted, or braided into a sheet or tube and cut into leaflet form. Appropriate cutting processes may include, but are not limited to, laser cutting, EDM, hot knife, die cutting, stamping, razor, scissors, or sharp blade. In some embodiments, PGSU fiber is used to create a single layer of woven or knitted fabric. In other embodiments, PGSU fiber is used to create a multilayered fabric such as pile and/or ground for a double needle bar knitted textile or orthogonal woven textile. In other embodiments, PGSU fiber is used to create a textured knitted or woven textile such as velour cloth. A velour architecture, for example, may impart anisotropy in mechanical properties and guide extracellular matrix (ECM) microstructure to better mimic native leaflets. In some embodiments, a combination of PGSU fiber and alternative biodegradable and/or nondegradable yarns forms a multiplex material textile to target varied strength, degradation, and flexibility properties.

In some embodiments, the PGSU in fiber form is then cut up for a non-woven construction, where the non-woven construction is formed as a sheet for leaflets. For example, chopped fibers may be used as a pile or in a fibrous mat. Fibrous architecture may impart anisotropy in mechanical properties and guide ECM microstructure to better mimic native leaflets.

In some embodiments the PGSU is provided as an electrospun mat. The PGSU electrospun mat may either serve as a standalone sheet to be cut into leaflet form via appropriate cutting processes including, laser cutting, EDM, hot knife, die cutting, stamping, razor, scissors, or sharp blade or directly electrospun onto a base substrate such as a textile or PGSU film. PGSU electrospun mat may provide a nano-filament architecture, enabling improved cellular response and tissue regeneration.

In some embodiments, leaflets are cut to shape from larger sheets of PGSU.

In some embodiments, a leaflet is molded directly around the annulus ring 14 of the heart valve frame 12, using reaction injection molding.

In some embodiments, a leaflet is attached to the heart valve frame 12 through other manufacturing or securing mechanisms during assembly. Such mechanisms may include, but are not limited to, crimping, welding, or suturing.

In some embodiments, a leaflet includes PGSU components with different degrees of crosslinking, or gradients of crosslinking, such as, for example, in different sections of the leaflet, to achieve desired mechanical properties and performance. For example, the leaflet may include a higher PGSU crosslinking density resulting in less flexibility toward a proximal end of the leaflet but a lower PGSU crosslinking density resulting in greater flexibility toward a distal end of the leaflet.

This multi-component or multi-crosslinking design may be molded as a single part, reducing manufacturing complexity, time, and cost. The properties of PGSU allow for such a manufacturing process, since its polyol-isocyanate mixing ratio can be tuned on the fly during mold filling. For example, meter-mix-dispense equipment used for PGSU reaction injection molding can alter the mixing ratio as material is dispensing into a mold, allowing designated cavities to be filled with specific material formulations in precisely dispensed volumes. Alternatively, meter-mix-dispense equipment may be used to fill designated portions of the mold, leaving some cavities empty. Once curing is complete in those regions, a different formulation is dispensed into the mold to fill designated open cavities. This leads to a multi-step, step-wise filling of the mold, with different formulations dispensed into the mold at each step, successively filling portions of the mold to completion. PGSU bonds well to itself, so the material filled in at a subsequent step chemically bonds with the already-cured material filled in at a previous step, so the final part is bonded together and not separate pieces of cured material.

In some embodiments, the PGSU of the synthetic leaflet not only initially provides the appropriate mechanical properties for the leaflet to open and close the heart valve replacement as an artificial heart valve but also serves as a scaffold such that as the PGSU degrades in vivo, it is replaced by generated connective tissue that allows the artificial heart valve to continue to function properly. The scaffold architecture and features may include porosity, channels, patterns, and roughness in one or more regions of the design. The scaffold architecture and features may be part of the PGSU design and/or may come from an underlying textile. In some embodiments, the PGSU scaffold may be formed to include one or more layers loaded with one or more biologics, active pharmaceutical ingredients, or porogens selected to promote connective tissue infiltration into the leaflet during biodegradation of the PGSU and subsequent release of the loaded material. In some embodiments, the leaflet is formed with varying crosslinking densities to promote connective tissue infiltration. In some embodiments, the leaflet is seeded with appropriate cells, preferably the patient's own autologous cells or alternatively donor allogeneic cells, prior to or at the time of implantation to promote connective tissue infiltration into the leaflet.

In some embodiments, an artificial heart valve includes a delivery vehicle to aid in delivery of the artificial heart valve to a target location. In some embodiments, the delivery vehicle is applied over at least a portion of the artificial heart valve. The delivery vehicle maintains at least a portion of the artificial heart valve in a contracted or collapsed state prior to delivery to the target location but permits the artificial heart valve to convert quickly to a three-dimensional (3D) expanded state upon reaching the target location that aids in retaining the artificial heart valve at the target location. In exemplary embodiments, the delivery vehicle quickly degrades at the target location to permit the artificial heart valve to convert to its 3D expanded state. In some embodiments, the delivery vehicle includes PGS.

Some artificial heart valves include a heart valve skirt that covers at least part of the heart valve frame 12. The heart valve skirt may include an inner skirt that covers the radially inner part of the heart valve frame 12 and an outer skirt that covers the radially outer part of the heart valve frame. In some embodiments, a PGSU coating is applied on the heart valve skirt of an artificial heart valve.

Some artificial heart valves include a wire form stent as part of the heart valve frame 12. In some embodiments, a PGSU coating is applied on the stent of an artificial heart valve.

In other embodiments, a PGSU overmold is applied on a leaflet component.

In other embodiments, a PGSU coating or overmold is applied on at least one leaflet component and at least one other heart valve replacement component simultaneously in a single process.

In other embodiments, standalone PGSU leaflets are formed by molding while a PGSU coating or overmold is applied on at least one other heart valve component in a single process. In such embodiments, the other heart valve component may include, but is not limited to, a skirt, posts, a stent, and/or an annulus ring 14.

In other embodiments, standalone PGSU leaflets, heart valve posts, and an annulus ring 14 are formed by molding while a PGSU coating or overmold is applied on at least one other heart valve replacement component in a single process. In such embodiments, the other heart valve component may include, but is not limited to, a skirt and/or a stent.

### EXAMPLES

The invention is further described in the context of the following examples which are presented by way of illustration, not of limitation.

PGSU films were fabricated by a draw down bar process using either a solvent-free PGS resin or a solvated pre-polymer solution at 80% w/w PGS concentration in acetone and propyl acetate.

PGSU films were fabricated by an open mold casting process using pre-polymer solutions at 15% w/w PGS concentration in acetone and propyl acetate.

The PGSU films were formulated at 2.5:1 PGS:HDI w:w ratio crosslinking for both the draw down bar process and the open mold casting process, with film thicknesses ranging from 150-350 μm.

The resulting PGSU films had an elastic modulus in the range of 12.1-16.6 MPa and an ultimate tensile strength in the range of 4.4-14.3 MPa.

Similarly fabricated PGSU films having thicknesses ranging from 150-350 μm and 3:1, 3.25:1, and 3.5:1 PGS:HDI w:w ratio crosslinking had an elastic modulus in the range of 7.3-11.3 MPa and an ultimate tensile strength of 3.0-6.9 MPa.

Formulations and processes using a PET release liner on one side and an open other side yielded a flat, smooth surface finish on both sides. In instances where a textile was embedded or coated with PGSU, all underlying topography from the textile was covered with a flat, smooth surface finish of PGSU.

All above-mentioned references are hereby incorporated by reference herein.

While the invention has been described with reference to one or more exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention but that the invention will include all embodiments falling within the scope of the appended claims. In addition, all numerical values identified in the detailed description shall be interpreted as though the precise and approximate values are both expressly identified.

What is claimed is:

1. A leaflet for a heart valve replacement, the leaflet comprising poly(glycerol sebacate) urethane.

2. The leaflet of claim 1, wherein the leaflet consists of poly(glycerol sebacate) urethane.

3. The leaflet of claim 1, wherein the leaflet comprises a leaflet substrate material and a coating on the leaflet substrate material, the coating comprising poly(glycerol sebacate) urethane.

4. The leaflet of claim 3, wherein the leaflet substrate material comprises a textile.

5. The leaflet of claim 3, wherein the coating conforms to the leaflet substrate material.

6. The leaflet of claim 3, wherein the coating has a smooth, flat surface obfuscating the substrate.

7. The leaflet of claim 1, wherein the poly(glycerol sebacate) urethane comprises poly(glycerol sebacate) urethane in fiber form.

8. The leaflet of claim 1, wherein the poly(glycerol sebacate) urethane is formed from a poly(glycerol sebacate) resin having a weight average molecular weight in the range of 14,000 Da to 20,000 Da.

9. The leaflet of claim 1, wherein the leaflet is shelf stable.

10. A heart valve replacement comprising a plurality of leaflets of claim 1.

11. The heart valve replacement of claim 10 further comprising a heart valve frame having a valve opening, wherein the plurality of leaflets extend from the heart valve frame to cover the valve opening.

12. The heart valve replacement of claim 10, wherein at least one of the plurality of leaflets comprises a first portion having a first crosslinking density and a second portion having a second crosslinking density different from the first crosslinking density.

13. The heart valve replacement of claim 10, wherein at least a portion of the heart valve replacement comprises a coating of poly(glycerol sebacate) urethane.

14. A process of forming a leaflet for a heart valve replacement, the process comprising:
casting a first solution comprising poly(glycerol sebacate) and isocyanate to form a leaflet composition comprising poly(glycerol sebacate) urethane; and
shaping the leaflet composition to form the leaflet.

15. The process of claim 14, wherein the casting forms a film of the leaflet composition.

16. The process of claim 14, wherein the process further comprises applying a second solution comprising poly(glycerol sebacate) and isocyanate as a coating overlying a layer formed by the first solution, wherein the second solution is different from the first solution, wherein the application comprises a process selected from the group consisting of casting, spray coating, dip coating, electrospinning, and electrospraying.

17. The process of claim 14, wherein the casting comprises reaction injection molding the solution to form the leaflet composition.

18. The process of claim 17, wherein the reaction injection molding comprises varying a ratio of poly(glycerol sebacate) to isocyanate during injection to vary a crosslinking density of the poly(glycerol sebacate) urethane in the composition.

19. The process of claim 14, wherein the casting comprises a process selected from the group consisting of closed molding and open molding.

20. The process of claim 14, wherein the casting comprises casting the first solution onto a leaflet substrate material to form a coating of the composition on the leaflet substrate material.

21. The process of claim 14, wherein the casting further comprises casting the first solution to form at least a portion of at least one other heart valve replacement component.

22. The process of claim 14 further comprising attaching the leaflet to a heart valve frame.

23. The process of claim 14, wherein the casting comprises casting the first solution around a heart valve frame of the heart valve replacement.

24. A process of forming a heart valve replacement, the process comprising:
providing a heart valve replacement substrate; and
applying a solution comprising poly(glycerol sebacate) and isocyanate to form a coating overlying at least a portion of at least one component of the heart valve replacement substrate, by a process selected from the group consisting of casting, spray coating, dip coating, electrospinning, and electrospraying;
wherein the coating comprises poly(glycerol sebacate) urethane.
